(19) United States
(12) Patent Application Publication
(10) Pub. No.: US 2021/0355073 A1
(43) Pub. Date: Nov. 18, 2021

(54) COPPER NANOPARTICLE BASED CHEMOSELECTIVE REDUCTION

(71) Applicant: Brown University, Providence, RI (US)

(72) Inventors: Shouheng SUN, East Greenwich, RI (US); Mengqi SHEN, Providence, RI (US); Hu LIU, Providence, RI (US)

(21) Appl. No.: 17/293,581

(22) PCT Filed: Nov. 15, 2019
(86) PCT No.: PCT/US2019/061776

§ 371 Date: May 13, 2021

Related U.S. Application Data
(60) Provisional application No. 62/767,931, filed on Nov. 15, 2018.

(51) Int. Cl. C07C 209/36; B01J 23/888; B01J 35/02

(52) U.S. Cl. CPC C07C 209/36; B01J 35/026; B01J 23/888

(57) ABSTRACT

The instant invention provides processes for a chemo selective reduction of a nitro group within a compound in the presence of other groups which can also be reduced. This aspect of the present invention provides an ammonia borane (AB) initiated chemoselective reduction process of a nitro group contained within a compound in the presence of a copper (Cu) nanoparticle based catalyst. The invention is also directed to Copper (Cu) nanoparticle (NP) based catalysts, selected from Cu/WOₓ, Cu/SiO₂, and Cu/C; wherein x represents an integer having a value of from about 2 to about 3.5, used in the chemo selective reduction of a nitro group contained within a compound in the presence of other groups which can also be reduced.

### STATEMENT REGARDING FEDERALLY SPONSORED RESEARCH

This invention was supported in part by the U.S. Army Research Laboratory and the U.S. Army Research Office under Grant No. W911NF-15-1-01. The government may have certain rights in the invention.

### BACKGROUND OF THE INVENTION

Selective reduction of functional aryl nitro (Ar—NO₂) to amine (Ar—NH₂) group is an important step towards preparation of a wide variety of organic compounds for pharmaceutical, polymer, herbicidal, and fine chemical applications. Nanosized transition metals/metal oxides have shown great potential as heterogeneous catalysts for the —NO₂ reduction because of their high activity, ease of separation and recyclability. However, what is difficult to achieve is selective reduction of Ar—NO₂ without effecting other reducible groups under the catalytic condition. In particular, the selective reduction of Ar—NO₂ over a vinyl group (Ar—C═C) in an aromatic compound remains to be challenging due to the uncontrolled hydrogenation of both Ar—NO₂ and Ar—C═C. Since Corma and Serna first reported that Au-based catalysts can show good selectivity in the reduction of 3-nitrostyrene in 2006, much progress has been made in using Au, Pt, Ag, Pd, and Ru catalysts for the selective reduction process. Recently, using non-noble metals to catalyze the same reaction also attracted much attention. Ni/TiO₂ showed 90% selectivity of 3-vinylaniline at 93% conversion under 450° C. and 15 bar of H_{2;} Fe₂O₃-based catalyst gave 93% selectivity on 100% conversion under 120° C. and 50 bar of H₂ after 16 h; and Co₃O₄-based catalyst was even more efficient at 110° C., 50 bar H₂, 6 h with 91% selectivity and >99% conversion. Despite the progress made in non-noble metal catalysts for the selective hydrogenation process, the catalytic reaction generally requires >100° C. temperature, high pressure of H₂ and hours of reaction time to complete.

Ammonia borane (AB) is regarded as a safe material for hydrogen storage (19.6 wt % hydrogen) and transport (solid at room temperature). In the presence of a transition metal catalyst, H₂ can be released from AB under ambient conditions, and AB has been used widely for hydrogenation reaction in milder reaction conditions to reduce nitrobenzenes to their amine derivatives. However, this hydrogenation process can become complicated when there exists a second reducible functional group in the aromatic structure as the competition between two reduction reactions often causes the reaction to be poorly selective.

Given the need for compounds wherein the nitro group can be selectively reduced and the difficulty in such selective reduction of the nitro group there needs to be a process which can afford the selective reduction of the nitro group with ease. The present invention affords such a process for the selective reduction of the nitro group.

### SUMMARY OF THE INVENTION

The instant invention provides processes for a chemo selective reduction of a nitro group within a compound in the presence of other groups which can also be reduced. This aspect of the present invention provides an ammonia borane (AB) initiated chemoselective reduction process of a nitro group contained within a compound in the presence of a copper (Cu) nanoparticle based catalyst. The invention is also directed to Copper (Cu) nanoparticle (NP) based catalysts, selected from Cu/WOₓ, Cu/SiO₂, and Cu/C; wherein x represents an integer having a value of from about 2 to about 3.5, used in the chemo selective reduction of a nitro group contained within a compound in the presence of other groups which can also be reduced.

This invention, in one aspect, is directed to Copper (Cu) nanoparticle (NP) based catalysts selected from Cu/WOₓ, Cu/SiO₂, and Cu/C; wherein x represents an integer having a value of from about 2 to about 3.5.

Another aspect of the invention provides processes for a chemo selective reduction of a nitro group within a compound in the presence of other groups which can also be reduced. This aspect of the present invention provides an ammonia borane (AB) initiated chemoselective reduction process of a nitro group contained within a compound in the presence of a copper (Cu) nanoparticle based catalyst.

### DETAILED DESCRIPTION OF THE INVENTION

This invention, in one aspect, is directed to Copper (Cu) nanoparticle (NP) based catalysts, selected from Cu/WOₓ, Cu/SiO₂, and Cu/C; wherein x represents an integer having a value of from about 2 to about 3.5. A preferred catalyst in this aspect of the invention is Cu/WOₓ. Another preferred embodiment provides a catalyst wherein x represents an integer having a value of from about 2.2 to about 3.0. Another preferred embodiment provides a catalyst wherein x represents an integer having a value of from about 2.5 to about 3.0, and an even further preferred embodiment provides a catalyst wherein x represents an integer having a value selected from 2.68, 2.71, 2.72, 2.79, 2.82, and 3.

Another aspect of the invention provides processes for a chemo selective reduction of a nitro group within a compound in the presence of other groups which can also be reduced. This aspect of the present invention provides an ammonia borane (AB) initiated chemoselective reduction process of a nitro group contained within a compound in the presence of a copper (Cu) nanoparticle based catalyst. A preferred embodiment of this aspect provides a chemoselective reduction process wherein the process is carried out in the presence of an aquatic solvent from about 10 minutes to about 30 minutes at a temperature of from about 10° C. to about 10° C. below the boiling point of the aquatic solvent. Another embodiment provides a process wherein the aquatic solvent is selected from water, aliphatic alcohol or a mixture thereof. The preferred aquatic solvent is water, an aliphatic alcohol selected from methanol, ethanol, iso-propanol, propanol, butanol, and iso-butanol. Yet another preferred embodiment provides a process wherein the mixture of the aquatic solvent is selected from any combination of water, methanol, ethanol, iso-propanol, propanol, butanol, and iso-butanol.

Another embodiment provides a process wherein the Cu nanoparticle based catalyst is selected from Cu/WOₓ, Cu/SiO₂, Cu/Al₂O₃, Cu/Ti₂O_{3,} Cu/CeO₂, Cu/graphene, Cu/graphene oxide, and Cu/C, wherein x represents an integer having a value of from about 2 to about 3. A preferred embodiment provides a process wherein x represents an integer having a value of from about 2.2 to about 3. A further preferred embodiment provides a process wherein x represents an integer having a value selected from 2.68, 2.71, 2.72, 2.79, 2.82 and 3. A particularly preferred embodiment provides a process wherein the Cu nanoparticle based catalyst is Cu/WO_{2.72}.

Another aspect of the present invention provides a chemoselective process for reducing a nitro group contained within a compound, said process comprising: (i) combining, in a sealed reaction vessel, a nitro group containing compound with an aquatic solvent to form a solution; (ii) diluting said solution with an aquatic dispersion of a Cu nanoparticle based catalyst to form a mixture; (iii) agitating said mixture in the sealed reaction vessel from about 10 minutes to about 30 minutes at a temperature of from about 10° C. to about 10° C. below the boiling point of the aquatic solvent; (iv) further diluting the agitated mixture in the sealed reaction vessel with a solution of Ammonia Borane (AB) in an aquatic solvent to yield a suspension; and (v) isolating the Cu nanoparticle based catalyst by subjecting the said suspension to centrifugation to yield a solution containing a compound wherein the nitro group is reduced to a corresponding amino group.

A preferred embodiment of this aspect of the invention provides a process wherein the aquatic solvent is selected from water, aliphatic alcohol, and a mixture thereof. A preferred aquatic solvent in one embodiment is water. Another preferred process the aquatic solvent is selected from aliphatic alcohol selected from methanol, ethanol, iso-propanol, propanol, butanol and iso-butanol. Yet another preferred aspect of the present invention provides a process wherein the aquatic solvent is a mixture of any combination of water, methanol, ethanol, iso- propanol, propanol, butanol, and iso-butanol.

Another preferred embodiment provides a process wherein the Cu nanoparticle catalyst is selected from Cu/WOₓ, Cu/SiO₂, Cu/Al₂O₃, Cu/Ti₂O_{3,} Cu/CeO₂, Cu/graphene, Cu/graphene oxide, and Cu/C, wherein x represents an integer having a value of from about 2 to about 3. A further preferred embodiment provides a process wherein the Cu nanoparticle based catalyst is Cu/WOₓ. Another preferred embodiment provides a process wherein x represents an integer having a value of from about 2.2 to about 3.0. A further preferred embodiment provides a process wherein x represents an integer from about 2.5 to about 3.0. A particularly preferred embodiment provides a process wherein x represents an integer having a value of 2.68, 2.71, 2.72, 2.79, 2.82, and 3. A particularly preferred embodiment provides a process wherein the Cu nanoparticle based catalyst is Cu/WO_{2.72}.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the certain specific methods and materials are described. As used herein, each of the following terms has the meaning associated with it in this section and as described elsewhere in this specification.

“About” as used herein when referring to a measurable value such as an amount, a temporal duration, and the like, is meant to encompass variations of ±20% or ±10%, more advantageously ±5%, even more advantageously ±1%, and still more advantageously ±0.1% from the specified value, as such variations are appropriate to perform the disclosed methods. As used herein, the term “DMSO” refers to dimethyl sulfoxide, the term “THF” refers to tetrahydro furan, the term “ODE” refers to 1-octadecene, the term “OAm” refers to oleylamine, the term “OAc” refers to oleic acid, the term “Cu(acac)₂ refers to Copper (II) acetylacetonate, Ni(acac) refers to nickel(II) acetylacetonate, and the term “OA” refers to oleic acid.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Certain specific examples include (C₁-C₆)alkyl, such as, but not limited to, ethyl, methyl, isopropyl, isobutyl, n-pentyl, n-hexyl and cyclopropylmethyl.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Certain specific examples include (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or poly-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “halo” or “halogen” alone or as part of another substituent means, unless otherwise stated, a fluorine, chlorine, bromine, or iodine atom, advantageously, fluorine, chlorine, or bromine, more advantageously, fluorine or chlorine. As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. In certain embodiments, aryl includes phenyl and naphthyl, in particular, phenyl.

As used herein, the terms “heterocycle”, “heterocyclyl”, “heterocyclic” or “hetero cycloalkyl”, by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “substituted” means that an atom or group of atoms has replaced hydrogen as the substituent attached to another group.

As used herein, the term “aquatic solvent” represents a solvent which is miscible with water and/or an aliphatic alcohol. Illustrative aquatic solvents, though not meant to limit, are water, aliphatic alcohols including methanol, ethanol, butanol, isobutanol, tertiary butanol, propanol, and isopropropanol. Other examples of aquatic solvents are dimethyl sulfoxide (DMSO), tetrahydrofuran (THF), dimethyl sulfoxide (DMF).

As used herein, the symbol “W” represents tungsten, “0” represents oxygen, “Si” represents silicon, “Cu” represents copper, “Al” represents aluminum, “Ar” represents argon, “Ti” represents titanium, “Ce” represents Cesium, and “C” represents carbon.

In this disclosure, “comprises,” “comprising,” “containing” and “having” and the like can have the meaning ascribed to them in U.S. Patent law and can mean “includes,” “including” and the liken “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 2 to 3 should be considered to have specifically disclosed sub-ranges such as from 2.01 to 2.99, from 2.1 to 2.9, from 2.5 to 2.8, etc., as well as individual numbers within that range, for example, 2, 2.3, 2.5, 2.6, 2.7, 2.72 and 2.8. This applies regardless of the breadth of the range.

### Experimental Details

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Chemicals and Materials

All reagents were used as received. Borane tert-butylamine complex (BBA, 97%), ammonia borane (AB) (NH₃BH₃, 90%), nitroarenes, copper (I) acetate (CuOAc, 97%), trioctylamine (TOA, 98%), tetradecylphosphonic acid (TDPA, 97%), oleylamine (OAm, >70%), oleic acid (OAc, 90%), 1-octadecene (ODE, 90%), and Nafion solution (5% in a mixture of lower aliphatic alcohols and water) were from Sigma-Aldrich. Copper (II) acetylacetonate (Cu(acac₂, 97%), palladium(II) acetylacetonate (Pd(acac₂, 99%), nickel(II) acetylacetonate (Ni(acac₂, 95%) and tungsten(IV) chloride (WC14, 97%) was purchased from Strem Chemicals. Hexane (98.5%) and ethanol (99%) were from Fisher Scientific. These chemicals were used without further purification. The deionized water was obtained from a Millipore Autopure System. Aquatic solvents used herein are as defined below. It is understood that a mixture of aquatic solvents can be used or a single aquatic solvent can be used as a solvent. Solvents not mentioned herein can function as an aquatic solvent as long as it is generally miscible with water or an alcohol.

### Experimental Section

Synthesis of Copper (Cu) nanoparticles (NPs): Cu NPs used herein were synthesized according to the procedures reported by Li Q., Fu J. J., Zhu W. L., Chen Z. Z., Shen B., Wu L. H., Xi Z., Wan T. Y., Lu G., Zhu J. J., Sun S. H., Tuning Sn-Catalysis for Electrochemical Reduction of CO₂ to CO via the Core/Shell Cu/SnO₂ Structure. J. Am. Chem. Soc. 2017, 139, 4290; Hung, L. I.; Tsung, C. K.; Huang, W. Y.; Yang, P. D., Room-Temperature Formation of Hollow Cu₂O Nanoparticles. Adv. Mater. 2010, 22, 1910; and Li, Q.; Zhu, W. L.; Fu, J. J.; Zhang, H. Y.; Wu, G.; Sun, S. H., Controlled assembly of Cu nanoparticles on pyridinic-N rich graphene for electrochemical reduction of CO₂ to ethylene. Nano Energy 2016, 24,1.

In a typical synthetic procedure for synthesizing Cu NP's in a four-neck flask, a mixture of 1 mmol copper(I) acetate, 0.5 mmol tetradecylphosphonic acid (TDPA) and 5 mL trioctylamine (TOA) was stirred and degassed at about 130° C. under an Argon (Ar) atmosphere for about 0.5 h. Then solution was heated to 180° C. and maintained at this temperature for 0.5 h. The solution was then heated to 250° C. over a period of about 5 mins in 5 min and held at temperature for 10 min to obtain 7 nm Cu NPs. The Cu NPs were separated by adding 100 mL ethanol and centrifuging at 8500 rpm for 8 min. The product was purified by dispersing in hexane and flocculating with ethanol, and precipitated by centrifugation (8500 rpm, 8 min). The purification process was repeated once, and the final Cu NPs product was re-dispersed into hexane for further use.

Synthesis of WO_{2.72} nanorodes (NRs): The WO_{2.72} NRs used herein were prepared according to the procedure outlined by Yu C., Guo X. F., Xi Z., Muzzio M., Yin Z. Y., Shen B., Li J. R., Seto C. T., and Sun S. H., in J. Am. Chem. Soc. 2017, 139, 5712.

In a typical synthesis, 0.25 mmol WC14, 5 mL ODE, 3 mL OAm were first mixed in a 20 mL vial through sonication to form a dark brown solution and transferred into a 100 mL four-neck round bottom flask. 10 mL ODE and 6 mL OAc were then added into the flask. The mixture solution was first heated to 20° C. in a gentle Ar flow and magnetic stirring for 0.5 h to remove air and moisture from the reaction system. The clear dark green solution was heated to 563 K in 0.5 h (when the reaction temperature reached 20° C., the Ar gas was switched to form an Ar blanket over the reaction system and the solution started to turn to blue). The reaction solution was kept at 290° C. for 10 h before it was cooled to room temperature. The product was separated from the solution by adding 80 mL ethanol and centrifuging at 9500 rpm for 8 min. Then 20 mL hexane was added to re-disperse the synthesized NRs and centrifugation (6000 rpm, 4 min) was applied to precipitate any undispersed. The product in the dispersion was then precipitated by adding 35 mL ethanol followed by centrifugation. The NRs were purified again with 15 mL hexane and 35 mL ethanol, and after separation by centrifugation, was dispersed in hexane for further use.

Synthesis of Cu/WO_{2.72}: 0.5 mmol Cu NPs, 1.0 mmol WO_{2.72} NRs, 16 mL OAm and 4 mL OAc were mixed into a 100 mL four-neck round bottom flask to form a mixture. Under a gentle argon flow, the mixture was quickly heated to 280° C. and was agitated for 0.5 hour before cooling to room temperature. The cooled mixture was washed by adding 100 mL ethanol followed by centrifuging at 8500 rpm for 8 min. The centrifuged mixture was purified by diluting with 20 mL hexane and 80 mL ethanol, followed by separation using centrifugation. The separated product was dispersed in hexane for further use.

Synthesis of Cu/C and Cu/SiO₂ NPs catalysts: The Cu/C and Cu/SiO₂ NPs were prepared according to the procedure outlined by Li, Q.; Zhu, W. L.; Fu, J. J.; Zhang, H. Y.; Wu, G.; and Sun, S. H., in Nano Energy 2016, 24, 1; and Metin, O.; Ozkar, S.; and Sun, S. H., in Nano Res. 2010, 3, 676.

The general procedure for synthesizing Cu/C and Cu/SiO₂ NPs catalysts involved suspending 10 mg of Ketjen carbon in 20 mL of hexane and sonicating said suspension for 1 h. The sonicated suspension was diluted by dropwise addition of 10 mg of Cu NPs in hexane. The resulting mixture was further sonicated for 1 h to ensure Cu NPs adsorption onto the carbon support. The Cu/C NPs were separated by centrifugation and washed with ethanol. This washing procedure was repeated three times. The Cu/C NPs were recovered and dried under vacuum. Cu/SiO₂ NPs catalysts was prepared by the same method.

Synthesis of CuNi NPs: The CuNi NPs were prepared according to the procedure outlined by Yu C., Fu J. J., Muzzio M., Shen T. L., Su D., Zhu J. J., and Sun S. H., in Chem. Mater. 2017, 29, 1413.

In a typical synthesis, 0.26 mmol of Cu(acac)₂, 0.26 mmol of Ni(acac)₂ were mixed with 7.5 mL of OAm, and 0.16 mL of OA under magnetic stirring in N₂ environment. The formed solution was heated to 110° C. and kept at this temperature for 0.5 h to remove moisture and oxygen from the reaction system. The reaction solution was further heated to 200° C. 0 and 160 mg of BBA dissolved in 1 mL of OAm was quickly injected into the solution. A visible color change from green to dark-brown was observed. The reaction was allowed to proceed for 40 min and cooled to room temperature. 100 mL ethanol was added, and the NP product was separated by centrifugation at 9000 rpm for 10 min. The NPs were washed twice with hexane/ethanol (v/v=1:15) and then stored in hexane for further use.

Synthesis of CuPd NPs: The CuPd NPs were prepared using the procedure outlined outlined by Xi Z., Li J. R., Su D., Muzzio M., Yu C., Li Q., and Sun S. H., in J. Am. Chem. Soc. 2017, 139, 15191.

In a typical synthesis, 0.25 mmol Cu(acac₂, 15 mL OAm, and 0.32 mL OAc were first mixed by magnetic stirring in a 50 mL four-neck flask and degassed under a gentle flow of argon at 343 K for 0.5 h resulting in a clear solution. Then 0.25 mmol Pd NPs dispersed in 2 mL hexane was dropped into the solution. The solution was heated to 280° C. and kept at 533 K for 1 h before it was cooled to room temperature. The CuPd NPs were separated by adding 100 mL ethanol and centrifuging at 9500 rpm for 8 min. The product was purified by dispersing in hexane and flocculating with ethanol, and precipitated by centrifugation (9500 rpm, 8 min). The purification process was repeated once, and the final NP (Cu₄₉Pd₅₁) product was redispersed into hexane for further use. d

Chemoselective hydrogenation reaction: Before the test, a two-neck round bottom flask (25 mL) containing a Teflon-coated stir bar was placed on a magnetic stirrer and thermostated to a desired temperature value. Then 3 mmol AB and 1 mmol nitroarenes were dissolved into 2 mL ethanol, respectively. The solution of nitroarenes compounds ethanol was added into the flask. Once the neck was connected to a balloon, and the other neck was sealed by rubber stopper. Afterwards, Cu-based catalysts were dispersed by sonication in ethanol (6 mL) and then transferred into the reaction flask. Next, the mixed solution was stirred with magnetic stirring for 15 min in the reaction flask. Finally, when the desired amount of AB was rapidly injected drop by drop into the flask using a syringe from the rubber stopper neck at a stirring rate of 800 rpm. After the experiment was over, the catalyst was separated from the suspension by centrifugation, and the yield of the product were determined by GC-MS with dodecane as the internal standard. The sample solution (0.2 μL) was directly injected into the GC-MS for quantitative analysis.

Cyclic voltammograms (CV) measurements: 20 mg of KetjenBlack EC-300-J carbon (C) was mixed with 20 mL of hexane, which was then sonicated for 0.5 h to form a uniform suspension. Then each of Cu/WO_{2.72} and Cu NPs in hexane was added into the suspension dropwise under sonication. After 1 h sonication, the C-supported powder was separated by centrifugation (8000 rpm, 8 min), washed with ethanol (three times), and dried at room temperature. Catalyst ink for electrochemical study was prepared by mixing the C-supported catalysts with 800 μL of ultrapure water, 200 μL of 2-propanol, and 10 μL of Nafion solution (5 wt %) for 1 h. 20 μL of catalyst ink was then deposited onto the glassy carbon rotating disk electrode (5 mm in diameter) for electrochemical measurements. An Autolab 302 potentiostat (Eco Chemie B.V., Holland) together with Pt wire as a pseudo-reference electrode and graphite bar as a counter electrode were used to obtain cyclic voltammetry (CV). CV measurement was scanning at room temperature between −1.0 V and 0.5 V at 100 mV/s in N₂-saturated CH₂Cl₂ solution (0.1 M Et₄NBF₄).

### Characterizations

Products obtained by various processes of the present invention were characterized bu using techniques known to one skilled in the art. Transmission electron microscopy (TEM) images were obtained using from a Philips CM20 operating at 200 kV. High-resolution TEM (HR-TEM) images were recorded with a JEOL 2100F transmission electron microscopy operated at an acceleration voltage of 200 keV. X-ray photoelectron spectroscopy (XPS) was performed on an ESCA 210 and MICROLAB 310D spectrometer using an Mg KR source. X-ray diffraction (XRD) patterns were collected on a Bruker AXS D8-Advanced diffractometer with Cu Ka radiation (λ=1.5418 Å). The inductively coupled atomic emission spectroscopy (ICP-AES) analyses were performed on a JY2000 Ultrace ICP atomic emission spectrometer equipped with a JY AS 421 autosampler and 2400 g/mm holographic grating. The analyses of products after chemoselective hydrogenation reaction were carried out by GC-MS using an Agilent 6890 GC coupled to a 5973 Mass spectrometer detector with a DB-5 (Agilent) fused silica capillary column (L×I.D. 30 m×0.25 mm, df 0.25 μm) and helium as carrier gas. The gas chromatograph was temperature programmed from 338 K (3 min initial time) to 573 K at 6 K/min (isothermal for 20 min final time). The mass spectrometer was operated in the electron impact mode at 70 eV ionization energy. Mass spectrometric data were acquired and processed using the GC-MS data system (Agilent Chemstation), and compounds were identified by gas chromatographic retention index and mass spectrum comparison with authentic standards, literature and library.

AB-initiated hydrogenation of nitrobenzene and styrene mixture in the presence of the Cu NP catalyst is represented by the reaction Scheme I below:

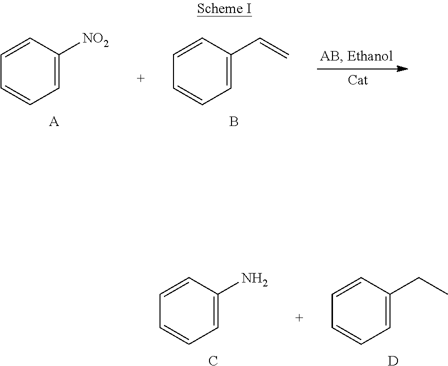

One would expect the nitro group in compound A to be reduced to the corresponding amine group yielding compound C, and the double bond in the styrene to be reduced to yield compound D. General reaction conditions and stoichiometry for the reaction shown in Scheme I are: Cu: 6 mol %; solvent: ethanol (10 mL); Internal standard: dodecane; Substrates: styrene (0.5 mmol); nitrobenzene (0.5 mmol).

Table S1 below lists the percent yield for the hydrogenation of the nitrobenzene and styrene mixture in Scheme-I using different Cu nanoparticle based catalysts. Chemo selective reduction using the Cu based nano particles is demonstrated by the fact that subjecting the mixture of the nitrobenzene and styrene to a reduction process only reduced the nitrobenzene to the corresponding amine (compound C) in excess of 99% while the double bond in the styrene molecule (compound B) was not reduced as evidenced by the fact that the process did not yield any amount of the corresponding compound D when the catalyst was Cu/WO_{2.72} and Cu/SiO₂ and a 5% yield when the catalyst was Cu/C.

| | | Yield/% | | | |
|---|---|---|---|---|---|
| Entry | Cat. | C | D | T/° C. | Time/h |
| 1 | Cu/WO_{2.72} | >99 | 0 | 25 | 1.5 |
| 2 | Cu/SiO₂ | >99 | 0 | 25 | 2.5 |
| 3 | Cu/C | >99 | 5 | 25 | 3 |

Scheme II below depicts the reduction of 3-nitrostyrene to 3-vinylaniline using various noble metal catalysts:

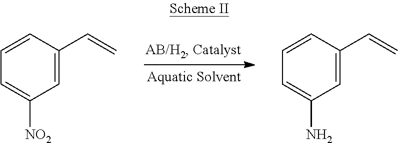

Table S2 below lists the yield for the chemoselective hydrogenation/reduction of 3- nitrostyrene to 3-vinylaniline over various non-noble metal catalysts

TABLE S2

| | | T | P | Time | Yield | Conversion |
|---|---|---|---|---|---|---|
| Catalyst | Reductant | (° C.) | (bar) | (h) | (%) | (%) |
| Cu/WO_{2.72} | AB | 25 | / | 1.5 | >99 | >99 |
| Cu/SiO₂ | AB | 25 | / | 2.5 | 98 | >99 |
| Cu/C | AB | 25 | / | 3 | 95 | >99 |
| Ni/TiO₂ | H₂ | 400 | 40 | 3 | 82 | 99 |
| Fe₂O₃-N/C | H₂ | 120 | 50 | 16 | 96 | 93 |
| Co₃O₄-L1/C | H₂ | 120 | 50 | 6 | 91 | 97 |
| CoO-Co₃O₄ | H₂ | 120 | 30 | 12 | 98 | 98 |
| Co₃O₄- | FA | 110 | / | 16 | 93 | 100 |
| NGr@C | | | | | | |
| Co NPs | H₂ | 120 | 50 | 15 | 82 | / |
| FeS₂ | H₂ | 120 | 50 | 18 | 100 | 100 |
| Co-Mo-S | H₂ | 250 | 11 | 8 | 88 | 95 |
| CoS₂ | H₂ | 110 | 30 | 7 | 98 | >98 |

*AB = Ammonia borane,
FA = Formic acid.

Table S2 above lists the yield for the reduction of 3 nitro styrene using various catalysts. As indicated in the table, reduction of 3-nitro styrene using a Cu nano particle based catalyst is carried out under milder conditions, compared to non-Cu nano particle based catalysts, (ambient temperature—25° C.), no gaseous pressure (p/bar), shorter reaction times (less than 3 hours) providing conversion in excess of 99% and a yield of over 95%.

Table S3 below lists the chemoselective hydrogenation of nitroarenes over Cu/WO_{2.72} catalyst in the presence of Ammonia Borane (AB) as a source of hydrogen, at temperatures ranging from ambient (25° C.) to about 60° C. The yield of the reduction process was 89% and above and the selectivity was about 99%.

| Substrate | Yield(%) | Product | Selectivity(%) | Condition |
|---|---|---|---|---|
| 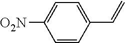 | 97 | 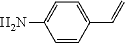 | 99 | 25° C., AB, 1.5 h |
| 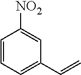 | 99 | 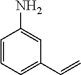 | 99 | 25° C., AB 1.5 h |
| 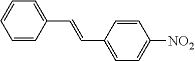 [SMILES: O=[N+]([O-])c1ccc(/C=C/c2ccccc2)cc1] | 95 | 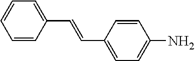 [SMILES: Nc1ccc(/C=C/c2ccccc2)cc1] | 99 | 50° C., AB, 3 h |
| 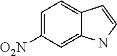 [SMILES: O=[N+]([O-])c1ccc2cc[nH]c2c1] | 80 | 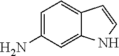 | 99 | 60° C., AB, 3 h |
| 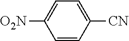 [SMILES: N#Cc1ccc([N+](=O)[O-])cc1] | 95 | 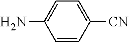 | 99 | 25° C., AB, 1.5 h |
| 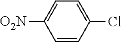 | 97 | 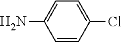 | 99 | 25° C., AB, 1.5 h |
| 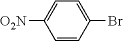 | 98 | 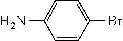 | 99 | 25° C., AB, 1.5 h |
| 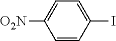 | 98 | 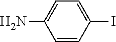 | 99 | 25° C., AB, 1.5 h |

Reaction conditions for the reduction of substrates listed in Table S3: Catalyst: Cu/WO_{2.72} (Cu: 6 mol %); ethanol (10 mL); Internal standard: dodecane; Substrates (1 mmol).

What is claimed:

1.-11. (canceled)

12. An ammonia borane (AB) initiated chemoselective reduction process of a nitro group contained within a compound in the presence of a copper (Cu) nanoparticle based catalyst, wherein the Cu nanoparticle based catalyst is selected from Cu/WOₓ, Cu/SiO₂, Cu/Al₂O₃, Cu/Ti₂O₃, Cu/CeO₂, Cu/graphene, Cu/graphene oxide, and Cu/C;
wherein x represents an integer having a value from about 2 to about 3.

13. The process of claim 12 wherein x represents an integer having a value of from about 2.2 to about 3.

14. The process of claim 13 wherein x represents an integer having a value selected from 2.68, 2.71, 2.72, 2.79, 2.82 and 3.

15. The process of claim 14 wherein the Cu nanoparticle based catalyst is Cu/WO_{2.72}.

16. A chemoselective process for reducing a nitro group contained within a compound, said process comprising:
(i) combining, in a sealed reaction vessel, a nitro group containing compound with an aquatic solvent to form a solution;
(ii) diluting said solution with an aquatic dispersion of a Cu nanoparticle based catalyst to form a mixture;
(iii) agitating said mixture in the sealed reaction vessel from about 10 minutes to about 30 minutes at a temperature of from about 10° C. to about 10° C. below the boiling point of the aquatic solvent;
(iv) further diluting the agitated mixture in the sealed reaction vessel with a solution of Ammonia Borane (AB) in an aquatic solvent to yield a suspension; and
(v) isolating the Cu nanoparticle based catalyst by subjecting the said suspension to centrifugation to yield a solution containing a compound wherein the nitro group is reduced to a corresponding amino group.

17. The process of claim 16 wherein the aquatic solvent is selected from water, aliphatic alcohol, and a mixture thereof.

18. The process of claim 17 wherein the aquatic solvent is water.

19. The process of claim 17 wherein the aliphatic alcohol is selected from methanol, ethanol, iso-propanol, propanol, butanol and iso-butanol.

20. The process of claim 17 wherein the mixture is selected from any combination of water, methanol, ethanol, iso-propanol, propanol, butanol, and iso-butanol.

21. The process of claim 16 wherein the Cu nanoparticle catalyst is selected from Cu/WOₓ, Cu/SiO₂, Cu/Al₂O₃, Cu/Ti₂O₃, Cu/CeO₂, Cu/graphene, Cu/graphene oxide, and Cu/C;
wherein x represents an integer having a value of from about 2 to about 3.

22. The process of claim 21 wherein the Cu nanoparticle based catalyst is Cu/WOₓ.

23. The process of claim 22 wherein x represents an integer having a value of from about 2.2 to about 3.0.

24. The process of claim 23 wherein x represents an integer having a value of from about 2.5 to about 3.0.

25. The process of claim 24 wherein x represents an integer having a value of from 2.68, 2.71, 2.72, 2.79, 2.82, and 3.

26. The process of claim 25 wherein the Cu nanoparticle based catalyst is Cu/WO_{2.72}.
